(19) United States
(12) Patent Application Publication
(10) Pub. No.: US 2011/0281366 A1
(43) Pub. Date: Nov. 17, 2011

(54) Systems and Methods of Detecting and Demonstrating Hair Damage Via Detection of Protein Loss

(76) Inventors: Michael Glenn Davis, Lebanon, OH (US); Stephen Worth Hendrix, Clarksville, OH (US)

(21) Appl. No.: 13/109,517

(22) Filed: May 17, 2011

Related U.S. Application Data
(60) Provisional application No. 61/345,321, filed on May 17, 2010.

(51) Int. Cl. G01N 33/68; B01J 19/00

(52) U.S. Cl. 436/86; 422/425

(57) ABSTRACT

Embodiments of a method for demonstrating hair damage comprises eluting protein fragments from a hair sample with an aqueous solution, adding a protein indicating reagent to the aqueous solution to provide a visual indicator corresponding to an amount of protein fragments eluted, and comparing the visual indicator to a scale to determine an amount of eluted protein fragments present in the aqueous solution.

### CROSS REFERENCE TO RELATED APPLICATION

This application claims the benefit of U.S. Provisional Application No. 61/345,321 filed May 17, 2010.

### FIELD OF THE INVENTION

Embodiments of the present disclosure are directed to a process for rapidly detecting protein damage in keratinous fibers, and are also directed to kits for detecting damage to hair proteins.

### BACKGROUND OF THE INVENTION

Hair damage through protein loss is a known problem; however, most people have no recognition of the amount of protein loss experienced by their hair, or their level of hair health in general. Protein loss may be caused by everyday occurrences and environmental factors such as UV ray exposure, bleaching, coloring, perming, straightening, mechanical manipulation, and salt water contact.

While all of the above mentioned factors lead to hair damage, each affects the hair architecture differently, thereby affecting the state of the hair (e.g., rendering the hair more brittle). The brittleness may be accompanied by a loss in substance, and can extend so far as to the breaking of hairs if they are subjected to damaging conditions on a regular basis.

The Peron et al. US Publication No. US 2006/0140893 (hereinafter “Peron”) discloses a process for detecting protein loss by contacting the hair with an extraction solution comprising a mixture of at least one of urea, thiourea, and derivatives thereof, with at least one reducing agent. The Peron process utilizes an extraction solution and a reducing agent to modify the protein structure by breaking the existing bonds in the keratinous fibers, and a reagent to detect the amount of protein loss.

It is clear that this technique tends to be restrictive in terms of implementation, cost, and procedure for ordinary consumers. Accordingly, there is a continual need for improved systems and methods to easily and accurately demonstrate a person's level of hair health that can be illustrated by visualizing the protein loss that can happen in, say, an ordinary shower or bathing situation and useful as a diagnostic for hair damage.

### SUMMARY OF THE INVENTION

The present disclosure relates generally to systems and methods for detecting and demonstrating hair damage by utilizing an aqueous solution to elute protein fragments from the hair without modifying the keratinous protein structure. Although the systems and methods of the present disclosure are not limited to particular protein indicating reagents or scales to assess the level of protein eluted, for the purposes of illustration, the method steps are described using particular reagents and scales.

In one embodiment, a method for demonstrating hair damage is provided, the method including eluting protein fragments from a hair sample with an aqueous solution, adding a protein indicating reagent to the aqueous solution to provide a visual indicator corresponding to an amount of protein fragments eluted, and comparing the visual indicator to a scale to determine an amount of eluted protein fragments present in the aqueous solution.

In another embodiment, a kit for demonstrating hair damage is provided, the kit including a protein indicating reagent capable of providing a visual indicator corresponding to the amount of protein fragments eluted from a hair sample in an aqueous solution and a scale to assess the quantitative and/or qualitative amount of protein fragments eluted in the aqueous solution. The scale allows comparison of the hair sample with a series of benchmarks associated with amounts of eluted protein fragments. The kit may also include instructions which inform a user to contact a hair sample with an aqueous solution.

In another embodiment, a method for demonstrating hair damage is provided. The method includes contacting a hair sample with an aqueous solution to elute protein fragments from the hair sample. The aqueous solution contains no solvents for keratinous proteins which act to break or reduce chemical bonds in the hair sample. The method also includes adding a protein indicating reagent to the aqueous solution to provide a visual indicator corresponding to an amount of protein fragments eluted, and comparing the visual indicator to a scale to determine an amount of eluted protein fragments present in the aqueous solution.

### DETAILED DESCRIPTION OF THE INVENTION

As used herein, “hair” means keratinous fibers of the human or animal origin, such as hairs on the head or eyelashes. Furthermore, as used herein, the term “keratinous protein” is understood to mean those proteins present in hair. As used herein, the term “protein fragments” means the amino acids and larger peptides that are damaged and broken off the keratinous protein structure and held within the hair structure by electrostatic interactions, weak hydrogen bonding matrix proteins and lipids, or any other force that does not include incorporation in the keratinous protein structure.

As used herein, “elutes,” “eluting,” and the like means removing proteins from hair via contacting hair with an aqueous solution without the addition of any reduction or extraction agents, thereby yielding no modification of the keratinous protein structure and no breaking or reduction of chemical bonds present in the hair sample other than electrostatic interactions, weak hydrogen bonding matrix proteins and lipids, or any other force that does not include incorporation in the keratinous protein structure.

As used herein, “elutable” means protein fragments present in the hair sample that may be removed from the hair structure in an aqueous solution without the addition of any reduction or extraction agents. Furthermore, “elutable” means proteins that may be carried out of the hair structure in an aqueous solution consisting essentially of water without the breaking or reduction of chemical bonds present in the keratinous protein structure other than electrostatic interactions, weak hydrogen bonding matrix proteins and lipids, or any other force that does not include incorporation in the keratinous protein structure.

In one embodiment, the method comprises providing a hair sample, eluting protein fragments from a hair sample with an aqueous solution, adding a protein indicating reagent to the aqueous solution to provide a visual indicator corresponding to an amount of protein fragments eluted, and comparing the visual indicator to a scale to determine an amount of eluted protein fragments present in the aqueous solution. The scale may be several shades of the same color or different colors to indicate different levels of protein present.

A hair sample may comprise a clipping of hair from the person to be tested. The number of hairs in a hair sample may vary depending on the characteristics of a person's hair, for example, the thickness of each individual hair. In one or more embodiments, the hair sample may comprise up to about 100 hairs, or from about 5 hairs to about 50 hairs, or from about 10 hairs to about 25 hairs. The length of hair included in the hair sample may vary. For example, it may be desirable to determine the protein loss at the root of the hair, at the tip of the hair, along the length of the hair, or a combination thereof. Thus, it is contemplated to remove hairs strands up to a couple feet in length. In other embodiments, the length of the hair strands in the sample may be up to about 10 cm, or from about 0.1 cm to about 5 cm, or from about 0.5 cm to about 2 cm.

Further referring to the eluting step, the hair sample may be contacted with an aqueous solution in a number of ways. In one embodiment, the hair sample may be submerged in a container filled with a predetermined amount of an aqueous solution. Alternatively, the hair sample may be inserted into a container, and then the container may be filled with a predetermined amount of aqueous solution. However, it is contemplated that the hair sample may also be contacted with an aqueous solution in a number of other ways, including, but not limited to rinsing, dipping, spraying, and soaking.

In one embodiment, the entire hair sample may be contacted with an aqueous solution. Alternatively, it is contemplated that only portions of the hair sample are contacted with the aqueous solution, such as the root or tip of the hair shaft. Upon addition to the solution, the hair sample may be agitated before, during, or after the introduction of the protein indicating reagent. The agitating step may comprise many different forms including shaking, stirring, inverting, adding additional solution, and other process steps not disclosed in this application.

The hair sample may be contacted with the aqueous solution for varying durations. In one embodiment, the contacting time is of sufficient duration to elute all or substantially all of the elutable protein fragments from the hair sample. Alternatively, the contacting time may be of sufficient duration to elute a majority of the elutable protein fragments. It is also contemplated that the hair sample may be contacted for other durations sufficient to elute other proportions of elutable protein fragments from the hair. The contacting time may range from about 30 seconds to about 60 minutes, or in specific embodiments, from about 30 seconds to about 5 minutes. However, other contacting durations are contemplated for use in the methods described herein. Generally, the elution of the protein fragments is obtained in a time period ranging from about 5 minutes to about 30 minutes, when the reaction is carried out at a temperature of about 25° C. It is also contemplated that stirring or shaking the aqueous solution, and/or heating the aqueous solution may increase the elution rate for the protein fragments.

In one embodiment, the aqueous solution contains no solvents for keratinous protein which act to break or reduce chemical bonds present in the keratinous protein of the hair sample. Solvents for keratinous proteins include, but are not limited to, reduction and extraction agents such as, for example, urea, thiourea, dithiothreitol, thioglycolic acid or thiolactic acid and their ester and amide derivatives, glyceryl monothioglycolate, cysteamine and its C₁-C₄ acylated derivatives, such as N-acetylcysteamine or N-propionylcysteamine, cysteine, N-acetycicysteine, thiomalic acid, pantethine, 2-3-dimercaptosuccinic acid, sulphites or bisulphites of an alkali metal or alkaline earth metal, N-(mercaptoalkyl)-co-hydroxyalkylamides, aminomercaptoalkylamides, derivatives of N-(mercaptoalkyl)succinamic acids and N-(mercaptoalkyl)succinimides, alkylaminomercaptoalkylamides, the azeotropic mixture of 2-hydroxypropyl thioglucolate and 2-hydroxy-1-methyl thioglycolate, mercaptoalkylaminoamides, and formamidinesulphinic acid derivatives. Additional materials contemplated for use with the solvent may include alkyl sulfates, alkylbenzenesulfonates, alkyl ether sulfates, alkylsulfonates, alkyl betaines, oxyalkylenated alkylphenols, fatty acid alkanolamides, oxyalkylenated fatty acid esters, and also oxyalkylenated fatty alcohols, and also oxyalkylenated fatty alcohols and alkylpolyglucosides.

Alternatively, the aqueous solution may consist essentially of water. In one embodiment, the aqueous solution may comprise any water type, for example, tap water, deionized water, distilled water, or combinations thereof. In addition, the aqueous solution may comprise additional compositions and additives that do not break or reduce the chemical bonds of the hair sample, including, but not limited to protein indicating reagents (e.g., colorimetric indicators), hair products, and salt. In one embodiment, the aqueous solution consists essentially of salt water, having a concentration of salt ranging from about 0 wt. % to about 25 wt. % by weight of the aqueous solution. However, it is also contemplated that the aqueous solution may comprise other additives and compositions not disclosed in this application.

The aqueous solution may be provided at a variety of temperatures to elute protein fragments from hair samples. Preferably, the aqueous solution may be provided at room temperature (about 20° C.). However, it is also contemplated that the aqueous solution be provided at a temperature above room temperature (about 20° C.). For example, the aqueous solution may be provided at a temperature ranging from about 20° C. to about 100° C., or from about 20° C. to about 35° C. However, it is also contemplated that the aqueous solution may also be provided at other temperatures suitable to elute protein fragments from hair samples. The aqueous solution may also be heated, for reasons of increasing the elution rate, to a temperature of greater than about 35° C., or a temperature of greater than about 70° C. It is understood that this temperature has to be compatible with the hair sample provided such that it elutes protein fragments from the hair, without destroying the keratinous protein. This heating may be applied by any conventional heating methods.

The amount of aqueous solution utilized may vary depending on many factors, including, but not limited to, the size of the hair sample, the amount of the protein indicating reagent, the size of the container, and the requirements of the user. Typically, in order to accommodate a sample of hair weighing about 50 mg, about 5 ml of aqueous solution is required. However, it is contemplated that a range of amounts of aqueous solution may be used in conducting the disclosed method. In one or more embodiments, the ratio by weight of the hairs to the aqueous solution ranges from about 0.2 mg/ml to about 100 mg/ml, or from about 1 mg/ml to about 50 mg/ml, or about 10 mg/ml.

In one embodiment, a protein indicating reagent may be added to the aqueous solution. Any protein indicating reagents suitable for visually identifying the eluted protein are contemplated. The protein indicating reagent may be brought into contact with the aqueous solution by introducing a predetermined amount of the reagent into the aqueous solution. The operation in which the protein indicating reagent is brought into contact with all or part of the aqueous solution may require the preliminary dilution of the protein indicating reagent.

In one embodiment, the protein indicating reagent may comprise a mixture of phosphotungstric acid and phosphomolybdic acid in phenol. Alternatively, the protein indicating reagent may comprise tetrabromophenol blue, a fluorescent dye, a Coomassie dye, or bicinchoninic acid. It is contemplated that one or multiple protein indicating reagents may provided to the aqueous solution in order to distinguish differing protein fragment levels present.

In another embodiment, the protein indicating reagent may be a solid, for example, in a desiccated form. Other solid forms for the protein indicating reagent are also contemplated, which include, but are not limited to, powders, tablets, and capsules. The amount of the protein indicating reagent may vary depending on the particular protein indicating reagent used, the form in which the protein indicating reagent is provided in, and the amount of aqueous solution utilized. In one embodiment, the protein indicating reagent may comprise a concentrated reagent to minimize the volume of the protein indicating reagent.

The method of detecting hair damage comprises comparing a visual indicator produced by the protein indicating reagent added to the aqueous solution with a scale to determine a qualitative and/or quantitative amount of eluted protein fragments present in the aqueous solution.

The protein indicating reagent may produce a visual indicator. The visual indicator provided by the protein indicating reagent may comprise many different signaling methodologies. In one embodiment, the visual indicator may yield a noticeable color change in the aqueous solution. It can also be the appearance of the color, modification of the color, or even the disappearance of the original color. Alternatively, the visual signal may comprise a change in transparency, texture, viscosity, or reflectivity of the aqueous solution such that one is able to distinguish varying levels of protein fragments present. In addition, other forms of visual indicators are also contemplated.

The scale may comprise a series of incremental protein loss values corresponding to predetermined visual indicators with known levels of protein fragments in order to assist the determination the protein loss of a hair sample. The scale may serve as a reference to determine the relative abundance of eluted protein fragments in the aqueous solution. The scale may be calibrated by a plurality of mixtures of protein fragments and non-keratinous proteins of predetermined concentrations. The scale may also comprise an arrangement of visible samples corresponding to the different levels of eluted protein fragments.

In one embodiment, the visual indicator may correspond to the level of protein fragments eluted from the hair sample. The intensity of the visual indicator may directly relate to the amount of protein fragments eluted, for example, the color of the aqueous solution may become more intense as the amount of the protein fragments eluted in the aqueous solution increases. However, the intensity of the visual indicator may also be inversely related to the amount of protein fragments eluted in the aqueous solution.

The scale may comprise a color chart, where the color chart comprises a plurality of colors or shades of a single color, wherein each color or shade corresponds to a qualitative and/or quantitative amount of eluted protein fragments. The amount of eluted protein fragments in the solution may be determined by comparing the visual indicator to the color chart, identifying the color that most closely corresponds to the visual indicator, and subsequently finding the concentration of eluted protein fragments. For example, a virgin hair sample may be treated with the method described herein, and compared to the color chart. Alternatively, a bleached hair sample may be treated with the method described herein, and compared to the color chart to determine an amount of protein loss. However, it is also contemplated that other types of hair samples may be treated with the method described herein, and compared to a color chart.

In another embodiment, the scale may comprise a series of benchmarked protein samples corresponding to various concentrations of eluted protein fragments. For example, the series of protein samples may be provided at concentrations ranging from about 0 μg/ml to about 200 μg/ml, with specific samples at concentrations of about 0 μg/ml, about 0.5 μg/ml, about 2.5 μg/ml, about 5 μg/ml, about 10 μg/ml, about 20 μg/ml, about 40 μg/ml, about 75 μg/ml, about 100 μg/ml, about 150 μg/ml, and about 200 μg/ml. Alternatively, the scale may comprise a series of benchmarked protein samples, wherein each sample corresponds to the protein loss associated with a particular hair treatment (not shown).

In another embodiment, the protein indicating reagent may be provided on a diagnostic test strip, in particular by adsorption or impregnation or coating with a solid support material, such as pH paper. The operation of adding the protein indicating reagent is then carried out by exposure and/or impregnation of the support material with all or part of the aqueous solution. The test strip provides a visual indicator upon insertion in an aqueous solution corresponding with the amount of protein fragments eluted from a hair sample in the aqueous solution. In one embodiment, the color of the test strip may be compared to a scale to provide a qualitative and/or quantitative amount of protein fragments eluted. After contacting the aqueous solution with the test strip, the test strip may be compared against a variety of types of scales. Alternatively, it is contemplated that the visual indicator disposed on the test strip 16 may be compared to a series of benchmarked solutions, or calibrated test strips provided corresponding to predetermined levels of protein fragments eluted from the solution.

As stated above, there are numerous factors which cause protein loss. Consequently, the scale may be specifically constructed to demonstrate typical protein loss values for specific factors such as bleaching, or the scale may be constructed to encompass typical protein loss values for all factors. For example, a sample corresponding to protein losses corresponding to each of one or more of the following may be provided: bleaching, dying, straightening, mechanical treatments, UV exposure, mechanical stressors, and repeated product applications. Without being bound by theory, higher concentrations of eluted protein fragments are present in the aqueous solution when the hair sample had been exposed to bleach, UV rays, mechanical stress, and salt water.

Other methods of evaluating the amount of protein fragments eluted from the hair sample are also contemplated. These methods include, but are not limited to spectroscopy, fluorospectroscopy, mass spectrometry, and gas chromatography.

In another embodiment, a kit for demonstrating hair damage is provided. The kit may comprise a protein indicating reagent capable of providing a visual indicator corresponding to the amount of protein fragments eluted in an aqueous solution, a scale to assess the amount of protein fragments eluted to an aqueous solution with no solvents for keratinous proteins which act to break or reduce chemical bonds present in the hair sample. The kit may include a container for immersing the hair sample, instructions, and other tools and devices necessary to conduct the method disclosed herein. The instructions may inform a user to contact a hair sample with an aqueous solution. The instructions may also inform a user to perform one or more of the following steps: adding a protein reagent to an aqueous solution; comparing a visual indicator to a scale to determine an amount of eluted protein fragments present in the aqueous solution; taking a hair sample; and agitating the aqueous solution. Optionally, the kit may also include an aqueous solution containing no solvents that break or reduce chemical bonds of keratinous protein present in the hair sample. In addition, the kit 20 may also include other components which facilitate the detection of protein loss from hair.

In one example, the amount of protein fragments eluted for virgin hair and bleached hair was compared using the above described method to demonstrate how much protein is lost due to damage of the keratinous protein. As used herein, “virgin” hair is hair that has not been subjected to the damaging factors described above, e.g., bleaching, UV exposure, salt water, etc. The results are provided in Table 1 below.

TABLE 1
Bleached and Virgin Hair Protein Fragment Eluted

| | Bleached Hair Sample | Virgin Hair Sample |
|---|---|---|
| Elution | Average Protein Fragments | Average Protein Fragments |
| time (min) | Eluted (μg/ml) | Eluted (μg/ml) |
| 5 | 130.16 | 10.14 |
| 10 | 171.67 | 16.92 |
| 15 | 206.50 | 21.8 |
| 20 | 227.78 | 29.68 |
| 30 | 246.82 | 37.18 |
| 40 | 279.26 | 48.16 |
| 50 | 314.28 | 57.37 |
| 60 | 341.84 | 60.10 |

In another example, the protein loss for several hair treatments was assessed using the method described herein. Particularly, the protein fragments eluted from virgin hair was compared to the protein fragments eluted from different types of bleached hair (i.e., H₂O₂, Persulfate pH 10, H₂O₂ pH 10) The results are provided in Table 2 below:

TABLE 2
Effects of Bleaching on Protein Fragments Eluted
Type of Bleaching

| Type of Hair | Average Protein Fragments Eluted (μg/ml) |
|---|---|
| Virgin Sample #1 | 60.66 |
| Virgin Sample #2 | 85.11 |
| H₂O₂/Persulfate pH 10 | 761.99 |
| H₂O₂ pH 10 | 370.95 |

In yet another example, several hair samples were analyzed for protein fragment eluted after being exposed to ultraviolet rays for various durations using the method described herein. The results are shown in Table 3 below.

TABLE 3
Protein Fragments Eluted from UV Exposure
Protein Fragments Eluted From Hair Increases with UV Exposure

| Hours of in-lab | Average Protein Fragments |
|---|---|
| UV exposure | Eluted (μg/ml) |
| 0 | 19 |
| 25 | 28 |
| 50 | 36 |
| 75 | 49 |

In another example, the protein fragment eluted from hair was assessed based on the different segments of hair using the method described herein. The results are provided in Table 4 below.

TABLE 4
Protein Fragments Eluted across the Hair Length
Root-to-Tip Differences

| Hair Segment | | Average Protein Fragments Eluted (μg/ml) |
|---|---|---|
| 0-2 | in. (root) | 48.73 |
| 6-8 | in. | 52.14 |
| 12-14 | in. (tip) | 136.52 |

The protein fragments eluted from hair samples was also assessed based on exposure to various types of water and bleaching agents. Table 5 shows the protein fragments eluted when different types of water are used in the method described herein. One sample included virgin hair samples contacted with de-ionized water. Another sample included virgin hair samples contacted with tap water. Yet another sample includes virgin hair contacted with salt water. Similar water treatments were also conducted in conjunction with bleached hair to compare the difference in protein fragments eluted.

TABLE 5
Protein Fragments Eluted Using Different Water Types
Salt Water Increases Protein Fragments Eluted

| | Average Protein Fragments |
|---|---|
| Hair Tested | Eluted (μg/ml) |
| Virgin Hair De-Ionized Water | 48.45 |
| Virgin Hair with Tap Water | 35.96 |
| Virgin Hair with Salt Water | 110.10 |
| Bleached Hair De-Ionized Water | 242.08 |
| Bleached Hair with Tap Water | 222.36 |
| Bleached Hair Salt Water | 492.33 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for demonstrating hair damage, the method comprising:
eluting a protein fragment from a hair sample with an aqueous solution;
adding a protein indicating reagent to the aqueous solution to provide a visual indicator; the visual indicator corresponding to a known amount of protein fragments eluted; and
comparing the visual indicator to a scale to determine an amount of eluted protein fragments present in the aqueous solution.

2. The method of claim 1, wherein the aqueous solution consists essentially of water.

3. The method of claim 1, wherein the scale comprises a series of benchmarked eluted protein fragment samples, wherein the series of benchmarked samples comprise samples ranging from known low to known high concentrations of eluted protein fragments.

4. The method of claim 1, wherein the scale comprises a color chart, where the color chart comprises a plurality of colors, wherein a color corresponds to a qualitative amount, quantitative amount, or both of eluted protein fragments.

5. The method of claim 4, wherein a color corresponds to the amount of protein fragment elution associated with a hair treatment.

6. The method of claim 1, wherein the aqueous solution is water at a temperature ranging from about 15° C. to about 35° C.

7. The method of claim 1, further comprising agitating the aqueous solution.

8. The method of claim 1, wherein the protein indicating reagent is provided on a diagnostic test strip, and wherein the diagnostic test strip yields a visual indicator corresponding to the amount of protein eluted in the solution.

9. The method of claim 1, wherein the protein indicating reagent comprises bicinchoninic acid.

10. The method of claim 1, wherein the protein indicating reagent comprises a mixture of phosphotungstric acid and phosphomolybdic acid in phenol.

11. The method of claim 1, wherein the protein indicating reagent comprises a Coomassie dye.

12. A kit for demonstrating hair damage, comprising:
a protein indicating reagent capable of providing a visual indicator; the visual indicator corresponding to an amount of protein fragments eluted from a hair sample in an aqueous solution;
a scale to assess the quantitative amount, qualitative amount or both quantitative and qualitative amounts of protein fragments eluted in the aqueous solution, wherein the scale allows comparison of the hair sample with a series of benchmarks associated with known amounts of eluted protein fragments; and
an instruction manual including a step to contact a hair sample with an aqueous solution.

13. The kit of claim 12, further comprising an aqueous solution containing no reduction or extraction agents.

14. The kit of claim 13, wherein the aqueous solution consists essentially of water.

15. The kit of claim 13, wherein the aqueous solution consists essentially of saltwater.

16. The kit of claim 12, wherein the protein indicating reagent is chosen from a group selected of phosphotungstric acid, phosphomolybdic acid, Coomassie dye, bicinchoninic acid, and combinations thereof.

17. The kit of claim 12, wherein the protein indicating reagent is provided in a desiccated form.

18. The kit of claim 12, wherein the protein indicating reagent is provided on a diagnostic test strip, wherein the color of the test strip corresponds to a certain amount of protein fragments eluted when compared against a scale.

19. The kit of claim 12, wherein the series of benchmarks comprises a color chart, wherein the color chart comprises a plurality of colors, wherein each color corresponds to a level of eluted protein fragments.

20. A method for demonstrating hair damage, the method comprising:
contacting a hair sample with an aqueous solution to elute protein fragments from the hair sample, wherein the aqueous solution contains no solvents for keratinous proteins which act to break or reduce chemical bonds in the hair sample;
adding a protein indicating reagent to the aqueous solution to provide a visual indicator corresponding to an amount of protein fragments eluted; and
comparing the visual indicator to a scale to determine an amount of eluted protein fragments present in the aqueous solution.
